(19) United States
(12) Patent Application Publication
(10) Pub. No.: US 2018/0321338 A1
(43) Pub. Date: Nov. 8, 2018

(54) ENDOENTERIC BALLOON COIL

(71) Applicant: UNIVERSITY OF UTAH RESEARCH FOUNDATION, Salt Lake City, UT (US)

(72) Inventors: John HADLEY, Centerville, UT (US); Dennis PARKER, Centerville, UT (US); Ronald CHAMBERLAIN, Stansbury Park, UT (US)

(21) Appl. No.: 15/773,529

(22) PCT Filed: Nov. 10, 2016
(86) PCT No.: PCT/US16/61294

§ 371 Date: May 3, 2018

Related U.S. Application Data
(60) Provisional application No. 62/254,126, filed on Nov. 11, 2015.
(60) Provisional application No. 62/256,538, filed on Nov. 17, 2015.

(51) Int. Cl. G01R 33/34; A61B 5/055; A61B 5/00

(52) U.S. Cl. CPC G01R 33/34084; A61B 2562/166; A61B 5/6853; A61B 5/055

(57) ABSTRACT

A catheter for use in magnetic resonance imaging includes a catheter shaft having a proximal end and a distal end. A flexible lumen is supported on the distal end of the shaft, and the flexible lumen is configured to be expanded and contracted using a fluid introduced via the proximal end of the catheter shaft. A magnetic resonance coil formed on the flexible lumen such that the magnetic resonance coil may expand and contract with the flexible lumen. The magnetic resonance coil is coupled to an external match and tune circuit via magnetic resonance imaging device. The balloon coil includes nested bazooka or sleeve baluns along the length of the cable to minimize common mode currents on the outer surface of the cable to prevent high current hot spots that cause heating of the cable.

### CROSS-REFERENCE TO RELATED APPLICATIONS

This application claims priority to co-pending U.S. Provisional Patent Application No. 62/256,538 filed on Nov. 17, 2015, and co-pending U.S. Provisional Patent Application No. 62/254,126 filed on Nov. 11, 2015, the entire contents of which are incorporated herein by reference.

### STATEMENT REGARDING FEDERALLY-SPONSORED RESEARCH OR DEVELOPMENT

This invention was made with government support under grant CA195453 awarded by the National Institutes of Health. The government has certain rights in the invention.

### BACKGROUND OF THE INVENTION

Imaging is indispensable in the identification, evaluation and follow-up of pancreatic and other lesions; however lesion classification based on imaging results alone is often difficult. Multi-detector computed tomography (CT) provides thin section scanning of the pancreas. CT and MRI are the primary modalities for initial detection and characterization of pancreatic lesions. However, many small lesions are not identified or accurately characterized using common CT and MRI techniques due to a lack of resolution.

FIG. 1 illustrates a balloon catheter 10 known in the prior art. The balloon catheter 10 includes a support wire 14 extending through a balloon 18. A rigid RF coil 22, contained within a housing 26, is attached to the catheter 10 (e.g., on a proximal end the support wire 14) and a distal end of the balloon 18. The RF coils 22 are not deformable, thus making it very difficult for a medical professional to maneuver the balloon catheter 10 in a patient. The lack of maneuverability detracts from the ability to properly rotate the coil 22 in order to obtain maximum signal sensitivity. In addition, the overall size of the balloon catheter 10 prevents easy introduction of the coil 22 into the gastrointestinal tract through the mouth of a patient, with or without sedation.

Commonly, RF coils such as rigid RF coil 22 have a match and tune circuit for optimizing coil performance (e.g., signal to noise ratio) that is mounted at a feed point of the coil loop in order to match the received MRI signal to a cable that then carries the signal to a preamp for amplification. The match and tune circuit increases the size of and complexity of the RF coil 22, but also requires that matching and tuning the RF coil must accomplished by making an inconvenient internal adjustment to the match and tune circuit within the RF coil to optimize signal to noise ratios (SNR) for specific anatomical locations.

Catheter deployable coils of many varieties have previously been developed for various MRI applications. Previous work has shown that internal RF coils, placed in the middle of large imaging volumes via catheter or other means, provide substantial improvements in SNR in the local region surrounding the inner coil. For example, Volland et al. showed that a small 2 cm diameter loop coil, placed directly on the surface of the heart in an in vivo open chest animal study, provided over 20 times greater SNR than state of the art external coil arrays, at the location of the heart wall. Some catheter-based coils employ medical balloons to facilitate the function or positioning of the coil. Larger diameter coils such as those used for endorectal prostate balloon coils appear to be best suited for pancreatic imaging. Larger loops have greater SNR penetration depth into the tissue at the expense of decreased SNR near the coil element.

Smaller diameter coils have very large SNR at the location of the coil with reduced penetration depth. Since it is not feasible to place an RF coil directly into the pancreas, optimal imaging of the pancreas requires positioning the RF coil as close as possible to the pancreas with a loop size large enough to provide useful signal sensitivity in the volume of the pancreas. The balloon catheter coil provides an attractive mechanism to position the coil near the pancreas in a mildly invasive way and the ability to expand the coil to a large diameter for increased SNR at the depth of the pancreas.

Previous balloon coils have used flexible or shape memory wire mounted on a balloon or between two balloons, or have used a rigid coil circuit mounted to the surface of a balloon. In addition, electronic circuits can be printed directly on balloons as sensors that are attached to the balloon using conductive inks or silicon based conductive material. The inks contain conductive particles that are congealed together through a heat based curing process to form a conductive surface on the balloon substrate.

Use of actual wires, fastened onto or between balloons, yields unstable coil tuning and matching characteristics during repeated expansion and contraction cycles of the balloon, because the wire is acting independently from the balloon. Wires that are consistent in shape are those formed from memory shape alloy such as those made from nickel and titanium (Nitinol) which cause large susceptibility artifacts near the wire and those that use some other mechanism, such as support fibers to help position the wire on the balloon, which are cumbersome to construct and can be inconsistent in loop positioning. Coils formed on a separate rigid substrate and attached to a balloon, such as the prostate coil, are not deformable (see FIGS. 1A and 1B). Rigid coil loops that are large enough to image the pancreas via the stomach or duodenum would be difficult at best to insert into the stomach or duodenum via the esophagus. Furthermore, even relatively small rigid coils are very difficult to maneuver and rotate (see FIGS. 1A and 1B). Coils printed on any substrate with conductive ink have lower conductivity than standard wire coils, resulting in greater coil resistance and reduced SNR. This problem is aggravated with small coils that are not sample noise dominated, meaning that the SNR depends more on the coil conductor noise than on the imaging sample noise.

### SUMMARY OF THE INVENTION

Endoenteric imaging has the ability to position the imaging sensor in the stomach or duodenum in close proximity to the pancreas. Conventional endoscopy requires sedation of the patient in order to tolerate the large caliber endoscope. In contrast, the coil according to embodiments of the invention can be mounted on a small flexible catheter about the same diameter as a conventional nasogastric tube. In some embodiments, patients may tolerate introduction of the coil without sedation. In other embodiments, patients may require some sedation to tolerate introduction of the coil.

Large phased array technology has made considerable progress in recent years. However, external arrays have fundamental SNR limitations that preclude low sensitivity applications such as DTI of deep structures in the abdomen such as the pancreas. Large phased array coils are sensitive to a large field of view and provide parallel imaging at the expense of decreased SNR. Maximum SNR is achieved by placing a small coil at the position of the imaging region of interest (ROI). A small coil at the position of the imaging ROI enables rapid imaging of a small field of view (FOV) with high SNR. Small internal coils can also be combined with external coil arrays to achieve large FOV imaging with improved image quality in the vicinity of the internal coil.

As the technical capabilities of CT and MRI have improved over the last several years, subcentimeter cystic lesions in the pancreas have been incidentally discovered with increasing frequency during abdominal imaging performed for unrelated reasons. Appropriate workup and treatment of the cystic pancreatic lesions is difficult. Endoscopic ultrasound and cyst aspiration can give clues to the nature of a cystic pancreatic lesion, but analysis of cyst fluid for CEA, mucin, and glycogen is not highly sensitive. Mucinous pancreatic lesions such as intraductal papillary mucinous neoplasm (IPMN) have malignant potential. The decision to resect IPMN depends on many factors, including crucial imaging findings. IPMN in the main pancreatic duct is typically resected due to high risk of malignancy. The determination of main duct vs. side-branch IPMN relies entirely on imaging findings such as dilation of the main duct. With current MRI and CT imaging methods, the discrimination of main-duct from side-branch IPMN is inaccurate. Criteria for resection of side branch IPMN are more complicated, and include critical imaging findings such as presence of mural nodules or adjacent mass. Because these resection criteria are based on imaging findings, their value depends entirely on the quality of imaging. By achieving higher resolution higher SNR depiction of pancreatic lesions, embodiments of the endoenteric MRI coil according to embodiments of the invention can improve the accuracy of radiologic assessment of cystic pancreatic lesions to determine the appropriateness of resection.

In addition, improved resolution and SNR in pancreas MRI could result in earlier detection of signs of malignancy in pancreatic lesions, as well as more accurate depiction of vascular invasion of pancreatic tumors for correct surgical planning. One embodiment of the invention relates to novel local RF coils that are deployed on an inflatable balloon that can be advanced into the stomach or duodenum mounted on a small caliber catheter and deployed next to the pancreatic head or body. These local coils can give better SNR and resolution than conventional MRI coils, for better characterization of pancreatic lesions. Successful completion of this project will enhance the ability of MRI to provide high-resolution MR images that better aid the clinician in visualizing, diagnosing, staging, and monitoring pancreatic lesions.

Specifically, high resolution imaging of the pancreas can enable 1) improved identification of marginally resectable cases, 2) identification of smaller lesions, and 3) characterization of cystic pancreatic lesions. The increased signal sensitivity also enhances the benefits of diffusion and perfusion weighted imaging, elastography, and may enable novel pulse sequences to be employed such as pharmacokinetic analysis of contrast agents or molecular imaging and spectroscopy for use in pancreatic lesion identification, characterization, and management. This technology could be used to image the tissues of the esophagus, the stomach, duodenum and pancreas.

Coils constructed on balloon catheters according to embodiments of the present invention have the potential to address the problems described above. They have a relatively small insertion diameter, and would be well tolerated by the patient. They are steerable and easily maneuvered into and out of spaces such as the duodenum. They are easily rotated to the desired orientation for maximum signal sensitivity in the region of interest. They can be made with a variety of balloon shapes and sizes that could accommodate the use RF coils of different shapes and sizes. These balloon coils can be adapted for essentially any internal imaging scenario where it is desired to place the coil close against a lumen wall or tissue surface. In particular, these coils are well suited for imaging of the esophagus, stomach pancreas and duodenum. Finally, using these same balloon coil construction techniques, multiple coils can be placed on the same balloon to form arrays that overcome single loop coil SNR performance limitations.

The present invention provides, in one aspect, a catheter for use in magnetic resonance imaging includes a catheter shaft having a proximal end and a distal end. A flexible lumen is supported on the distal end of the shaft, and the flexible lumen is configured to be expanded and contracted using a fluid introduced via the proximal end of the catheter shaft. A magnetic resonance coil formed on the flexible lumen such that the magnetic resonance coil may expand and contract with the flexible lumen.

The invention provides, in another aspect, magnetic resonance imaging system comprising a magnetic resonance imaging device and a catheter including a magnetic resonance coil that is coupled to the magnetic resonance imaging device. The catheter includes a catheter shaft having a proximal end and a distal end. A flexible lumen is supported on the distal end of the shaft, and the flexible lumen is configured to be expanded and contracted using a fluid introduced via the proximal end of the catheter shaft. A magnetic resonance coil formed on the flexible lumen such that the magnetic resonance coil may expand and contract with the flexible lumen. The magnetic resonance imaging device includes a match and tune circuit that is disposed remotely from the magnetic resonance coil.

The invention provides, in yet another aspect, a method for magnetic resonance imaging of anatomical locations within a patient. The method includes inserting a catheter into the patient, where the catheter includes a flexible lumen supported on a distal end of the catheter that is configured to be expanded and contracted using a fluid introduced via the proximal end of the catheter shaft. The catheter further includes a magnetic resonance coil formed on the flexible lumen. The flexible lumen is contracted during insertion of the catheter. The method also includes locating the catheter to a desired anatomical location within the patient, expanding the flexible lumen, and performing magnetic resonance imaging using a magnetic resonance imaging system.

Embodiments of the present invention provide for a catheter-deployable medical-balloon-substrate RF coil that can be collapsed and expanded for positioning in the stomach or duodenum. In addition, embodiments of the present invention provide for the use of conductive ink to form an initial coil trace seed layer on a collapsible surface such as a flexible medical balloon and then electroplating the resulting moderately conducting surface to form a highly conducting, low-resistance (high Q) RF coil trace which is required by small loop MRI coils to minimize unwanted noise and improve coil signal sensitivity.

Other features and aspects of the invention will become apparent by consideration of the following detailed description and accompanying drawings.

### BRIEF DESCRIPTION OF THE DRAWINGS

FIG. 1A is a side view of a balloon catheter with RF coil for MRI known in the prior art.

FIG. 1B is a front view of a balloon catheter with RF coil for MRI known in the prior art.

FIG. 2 is a perspective view of an exemplary RF balloon coil in an inflated state.

FIG. 3 is a perspective view of the RF balloon coil in a deflated state.

FIG. 4 is a schematic diagram of the RF balloon coil coupled to an MRI device.

FIG. 5 is a photograph of a set of RF coils used for testing.

FIG. 6 is a graph of relative signal to noise ratio vs. distance including plots for the coils shown in FIG. 5.

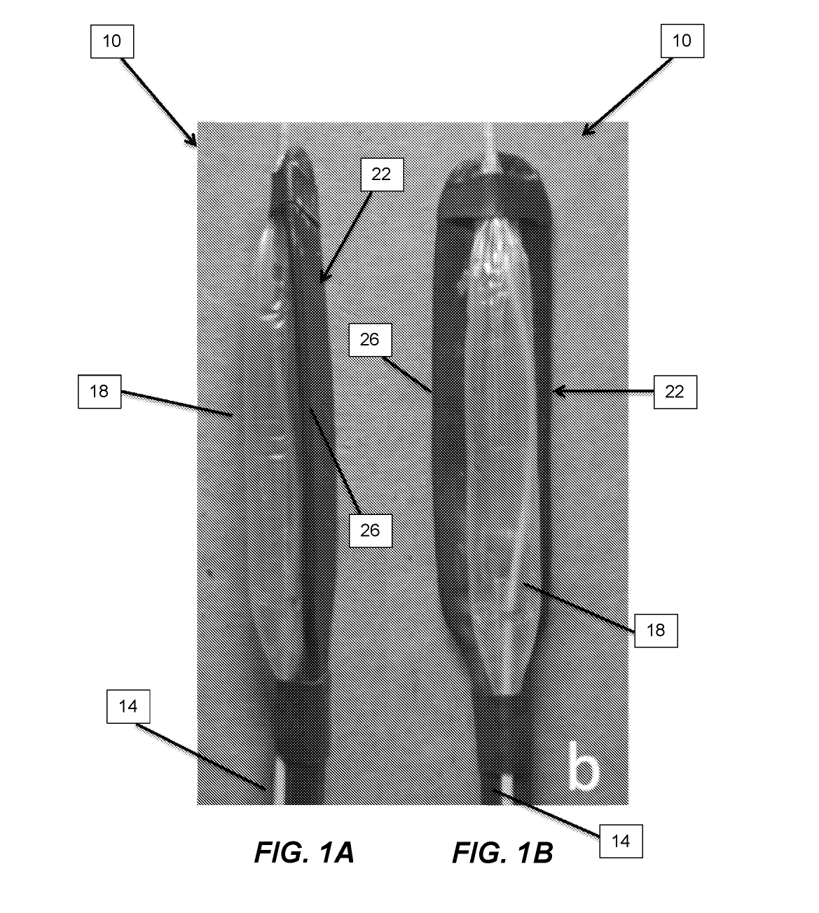

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting.

### DETAILED DESCRIPTION

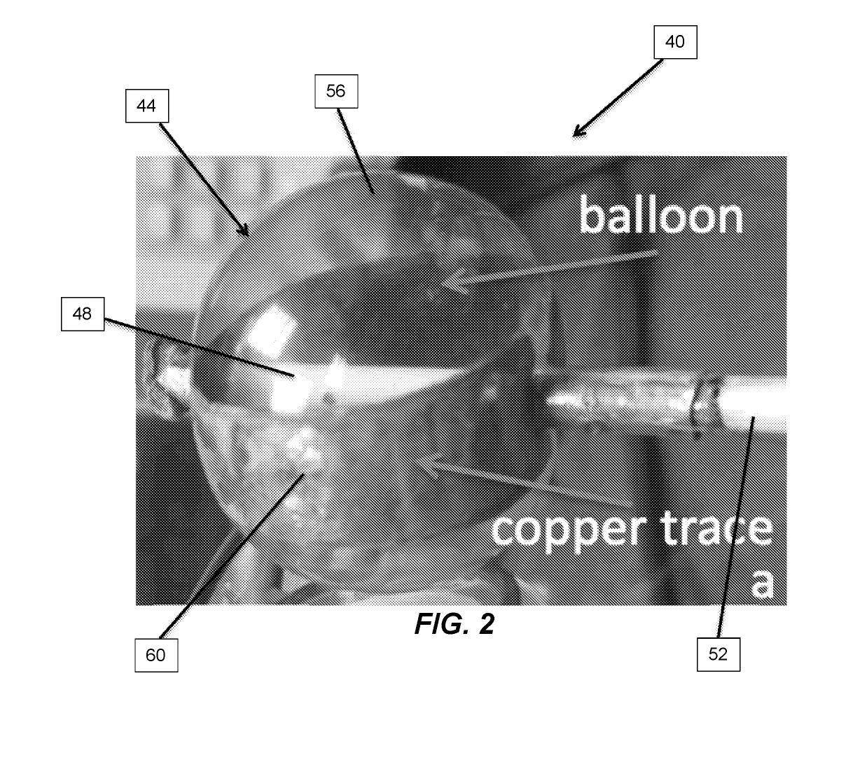

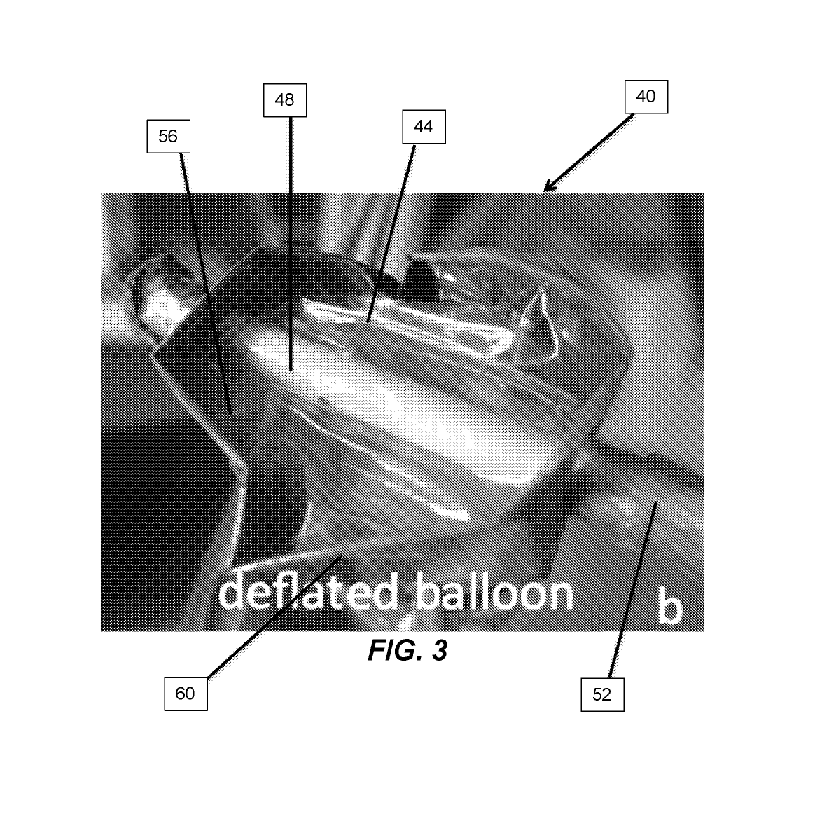

FIGS. 2 and 3 illustrate a catheter-based radio frequency (RF) balloon coil or balloon coil 40 according to an embodiment of the present invention. The balloon coil 40 is configured to be inserted into a patient (e.g., into the gastrointestinal (GI) tract) and is used to acquire high resolution MRI images (i.e., high signal to noise ratio (SNR)) by achieving high sensitivity and rapid image acquisition speed. The balloon coil 40 may be used in MRI imaging of various locations within the human body, but is particularly useful for imaging the pancreas and upper GI tract (i.e., esophagus, stomach, duodenum, etc.). As a result of the high resolution MRI images obtained when using the balloon coil 40, health professionals may identify very small lesions that may otherwise not be identified in standard MRI processes. The increased resolution also allows health professionals to more accurately characterize lesions identified in MRI images. In addition, due to the high resolution imaging, the balloon coil 40 has been contemplated as useful in Focused Ultrasound (FUS) treatments. Specifically, FUS treatments could be monitored for a number of factors, such as temperature.

With continued reference to FIGS. 2 and 3, the balloon coil 40 includes a balloon 44 supported on a distal end 48 of a catheter shaft 52. The balloon 44 is defined by a flexible lumen 56 delimiting an inflatable interior volume and an exterior surface. The exterior surface supports a thin, flexible metal 60 (i.e., copper, silver, etc.) that is generally deformable with the flexible lumen 56. In the illustrated embodiment, the metal 60 forms a single, generally continuous strip extending radially around the lumen 56. However, other suitable patterns and formations (e.g., arrays) may be used. In some embodiments, a second balloon may be placed over the balloon 44 as a protective sheath to provide, among other things, water proofing and abrasion protection for the balloon coil 40.

The catheter shaft 52 includes a body extending from a proximal end (not shown) to the distal end 48 of the shaft 52. The proximal end includes a fluid inlet fluidly coupled to a fluid outlet, located on the distal end 48 of the shaft 52 within the interior volume of the lumen 56, by a conduit extending through an interior of the body. The conduit allows a user to introduce or remove a fluid (i.e., air, saline solution, etc.) through the inlet in order to inflate (FIG. 2) and deflate (FIG. 3) the balloon 44. Inflation is generally accomplished after the distal end has been inserted into a patient.

As noted above, RF coils for use with MRI devices include a match and tune circuit that is coupled at or near a feed point of a loop of the coil in order to match the received MRI signal to a cable (e.g., a coax cable) that carries the MRI signal to a preamp of the MRI device for amplification. The balloon coil 40, however, is configured to be inserted into a patient. Therefore, a match and tune circuit that results in an increase in size or complexity of the balloon coil 40 may be disadvantageous in certain embodiments.

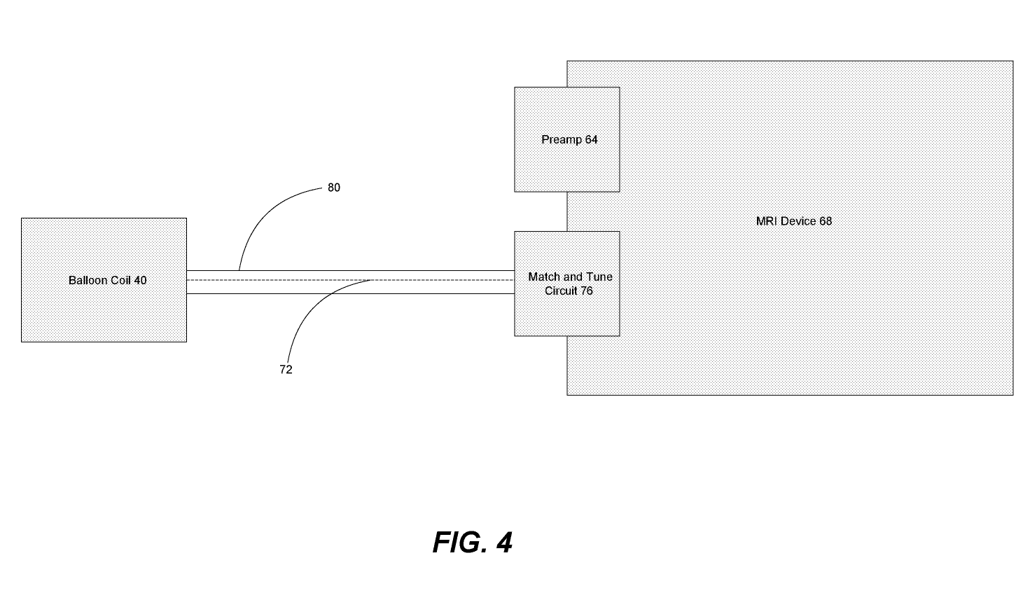

In the embodiment illustrated in FIG. 4, the balloon coil 40 is configured to be coupled to a preamp 64 of an MRI device 68 via a half wavelength cable 72 such a match and tune circuit 76 may be positioned remotely from the balloon coil 40 (e.g., external to the patient and/or proximate to the preamp) thereby reducing the overall size and complexity of the balloon coil 40. This configuration enables tuning and matching of the balloon coil 40 to any anatomy the balloon coil 40 may be used for because it enables external tuning and matching after the balloon coil 40 has been positioned within a patient. In addition, the cable 72 is in electrical isolation along the entire length of the cable 72 such that risk of electrical shock and arcing (i.e., RF burns) are mitigated.

In the embodiment described above, the length of the half wavelength cable 72 may pose difficulties for operations. For example, during the transmit portion of an MRI pulse sequence, RF power deposition may be negatively affected by an outer shield of the cable 72 becoming an antenna that picks up transmit power from a transmit coil of the MRI device resulting in hot spots along the cable 72. Hot spots along the cable could potentially reach temperatures that would damage tissue of a patient that is adjacent to the cable 72.

To counteract this potential downfall, the balloon coil 40 includes nested bazooka or sleeve baluns 80 along the length of the cable to minimize common mode currents on the outer surface of the cable 72 to prevent high current hot spots that cause heating of the cable 72.

Two methods of constructing the balloon coil 40 have been specifically contemplated, although other construction methods are possible. The first involves constructing a metal loop on the balloon substrate using an electroless plating bath followed by an electroplating process to deposit metal 60 onto the exterior surface of the lumen 56. In this process, the entire exterior surface is plated with metal, and then unwanted metal is removed using an etching process. In one example, the etching process includes using ferric chloride to remove metal 60 from the balloon 44. However, other metal removal processes have been contemplated as useful. Then, the metal 60 is tuned to an MRI acceptable frequency of approximately 123 MHz using tuning wires attached to the loop so the coil is functional for MRI.

As seen in FIGS. 2-3, this construction method yields a balloon coil 40 that provides high coil conductivity while also providing a high degree of balloon coil 40 flexibility. FIG. 2 illustrates the balloon coil 40 in an inflated state, in which the metal 60 is generally continuous with the lumen 56 of the balloon 44. FIG. 3 illustrates the balloon coil 40 in a deflated state, in which the metal 60 has deformed with the lumen 56 as the balloon 44 was deflated such that the overall profile of the balloon coil 40 is greatly reduced. This allows a medical professional to easily introduce the balloon coil 40 into a patient while also providing a high degree of maneuverability for placing and orienting the balloon coil 40.

In a second method of constructing the balloon coil, a conductive ink (e.g., silver containing ink, etc.) is applied to the exterior surface of the lumen 56 in a pattern corresponding to the desired metal geometry. The ink is then cured to form a conductive surface lumen 56.

In one embodiment, the balloon 44 is subsequently electroplated such that metal 60 (e.g., copper, etc.) is deposited on the patterned conductive ink thereby increasing conductivity. The metal 60 is deposited such that it forms a thick enough layer to be operational as an MRI coil, yet flexible enough to be collapsible with the balloon lumen 56. Next, each of the coils will be tuned to acceptable MRI frequencies.

One advantage of the conductive ink lies in the fact that a specific pattern may be applied to the lumen and metal may be electroplated only to that surface. This negates the tedious etching process described above. In addition, this process also aids in optimizing balloon coil characteristics, such as metal thickness, balloon coil flexibility, and balloon coil SNR capabilities.

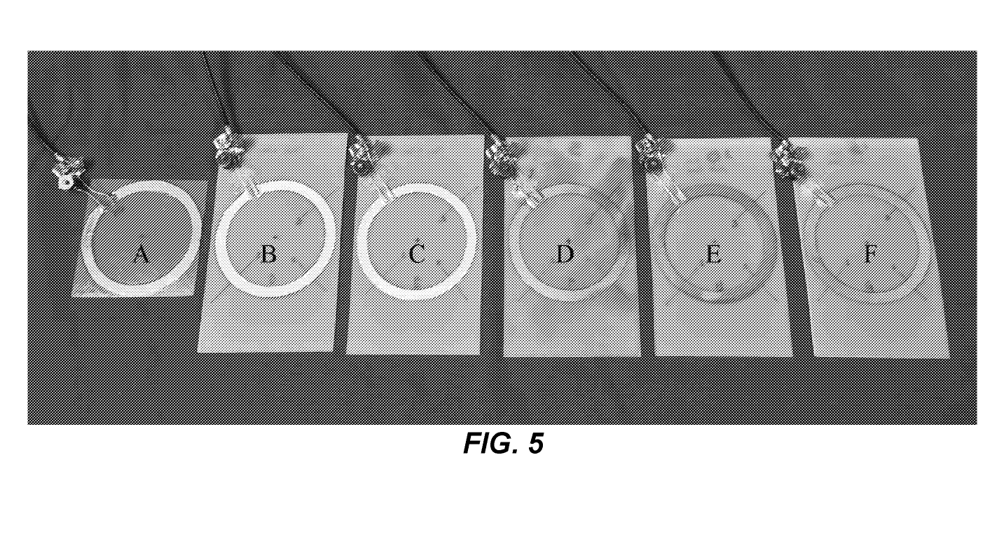

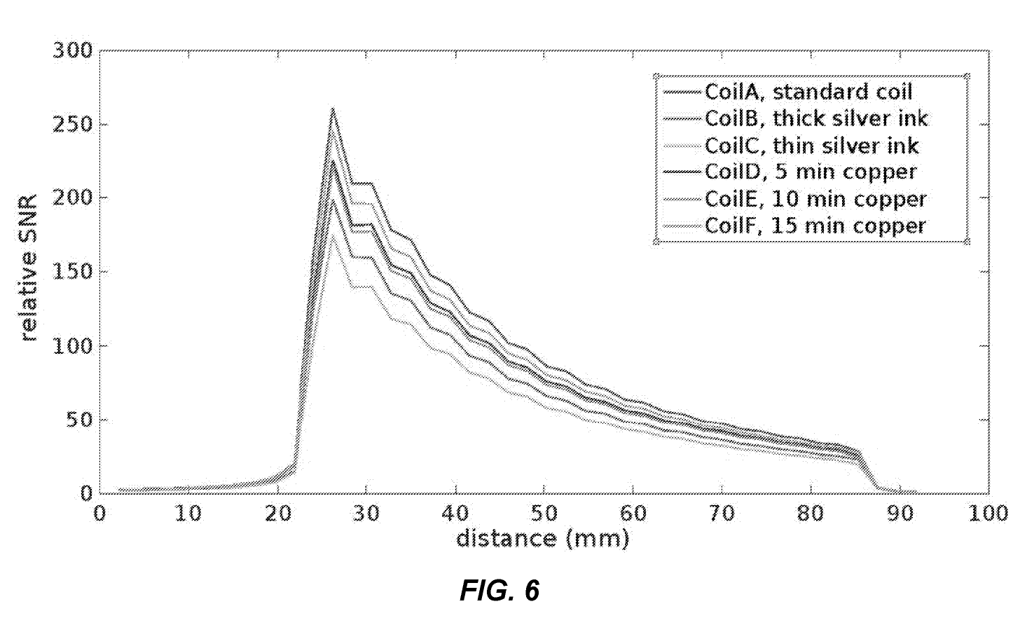

Testing has revealed the ability of silver conductive ink, and electroplated conductive ink, to be an effective radio frequency MRI coil. In an exemplary study, illustrated in FIGS. 5 and 6, six coils were evaluated with all imaging done on a Siemens Tim Trio 3T MRI Scanner. All coils were constructed on fiberglass formers as 52/62 mm inner/outer diameter coils with a single gap. The comparison standard (Coil-A, see Tablel below for characteristics) was solid copper. Coils B and C were thick and thin silver ink traces (Creative Materials Inc., 120-07), respectively, without plating. Coils D-F were thin silver ink traces that were copper plated for 5, 10 and 15 minutes, respectively. All loops were coated with varnish to keep the thin copper layers from oxidizing. 18-gauge tinned-copper wire leads and a tune/match circuit were positioned at the gap (FIG. 5). Leads were connected to Coil-A with solder and with silver ink for all other loops. Silver ink traces were cured at 130° C. for 5 minutes. Electrode copper plating was performed for coils D-F after an acid wash at 0.5 volts. The DC resistance was measured for each loop before circuitry was added. Each loop was tuned and matched at 123 MHz with an insertion loss better than −35 dB. Active and preamp detuning were better than −35 dB and −20 dB, respectively. SNR measurements were made using standard GRE sequences (TR/TE/flip/FOV=500 ms/4 ms/90°/280 mm, 128×128 matrix). SNR plots were constructed by averaging 5 image pixels through the axis of the coil, over 5 different scans for each coil.

TABLE 1

| | ink | plating | copper | | | |
|---|---|---|---|---|---|---|
| | thickness | time | thickness | ohms | | |
| coil | (μm) | (min) | (μm) | (DC) | rSNR | description |
| A | 0 | 0 | 35 | 0.3 | 209.3 | standard copper |
| | | | | | | coil² |
| B | ~15¹ | 0 | 0 | 1 | 159.5 | thick silver ink³ |
| C | ~6 | 0 | 0 | 1.7 | 139.7 | thin silver ink⁴ |
| D | ~11 | 5 | ~3 | 0.7 | 181.8 | thin ink, 5 min |
| | | | | | | plate⁵ |
| E | ~7 | 10 | ~6 | 0.7 | 177.1 | thin ink, 10 min |
| | | | | | | plate |
| F | ~6 | 15 | ~9 | 0.7 | 195.7 | thin ink, 15 min |
| | | | | | | plate |

¹Approximate silver ink and copper thickness was measured by averaging measurements at 3 different positions on the loop using a hand held micrometer.
²Copper etched on 1 oz. copper clad Kapton substrate (Dupont FR9150).
³Thick silver ink trace constructed using masking tape mask and squeegeeing the ink with a plastic ruler.
⁴Thin silver ink trace constructed using a masking tape mask and painting the trace with a small paintbrush.
⁵Plating voltage was set at 0.5 volts.

Results from this study show that the silver ink used can be electroplated with copper and, although the plating only occurs on one side of the silver trace, the electrical conductivity of the loop does increase. Silver ink thickness, plated copper thickness and DC resistance measurements are presented in Table 1. In addition, Table 1 shows example relative SNR measurements from ROIs near the coil. FIG. 6 shows the relative SNR results for the 6-coil comparison. As expected, these plots show the significant difference in SNR between the solid copper loop and the ink loops. They also demonstrate how conductivity is increased with copper plating. Results for Coil-E were not expected since its copper thickness would indicate a resulting SNR between those of Coil-D and Coil-F. Although every effort was made to keep the coil tune and match properties consistent, the loops were very sensitive and there may have been some unresolved problem with the silver ink wire attachments for Coil-E during the SNR measurements.

This work demonstrates that copper plating of silver ink coils is possible and it indicates that significant improvements in coil trace conductivity can be achieved. Consequently, the SNR performance of silver ink coils that have been plated with copper improves over silver ink coils without plating.

Although the invention has been described in detail with reference to certain preferred embodiments, variations and modifications exist within the scope and spirit of one or more independent aspects of the invention as described.

1. A catheter comprising:
a catheter shaft including a proximal end and a distal end;
a flexible lumen supported on the distal end of the shaft, the flexible lumen configured to be expanded and contracted using a fluid introduced via the proximal end of the catheter shaft; and
a magnetic resonance coil formed on an exterior surface of the flexible lumen.

2. The catheter of claim 1, wherein the magnetic resonance coil deforms to expand and contract with the flexible lumen.

3. The catheter of claim 1, wherein the magnetic resonance coil is at least partially formed from a conductive ink.

4. The catheter of claim 3, wherein the magnetic resonance coil is formed from a metal coupled to the conductive ink.

5. The catheter of claim 1, wherein the proximal end is disposed externally to a patient.

6. The catheter of claim 5, wherein the proximal end is coupled to a match and tune circuit of a magnetic resonance imaging device.

7. The catheter of claim 1, wherein the magnetic resonance coil is coupled to a magnetic resonance imaging device via a cable including at least one balun.

8. A magnetic resonance imaging system comprising:
a magnetic resonance imaging device;
a catheter including a magnetic resonance coil that is coupled to the magnetic resonance imaging device, the catheter including
a catheter shaft including a proximal end and a distal end;
a flexible lumen supported on the distal end of the shaft, the flexible lumen configured to be expanded and contracted using a fluid introduced via the proximal end of the catheter shaft; and
the magnetic resonance coil formed on an exterior surface of the flexible lumen;
wherein the magnetic resonance imaging device includes a match and tune circuit that is disposed remotely from the magnetic resonance coil.

9. The magnetic resonance imaging system of claim 8, wherein the match and tune circuit is disposed externally to a patient when the magnetic resonance imaging system is being operated.

10. The magnetic resonance imaging system of claim 8, wherein the magnetic resonance coil is coupled to the match and tune circuit via a cable that includes at least one balun.

11. The magnetic resonance imaging system of claim 8, wherein the magnetic resonance coil deforms to expand and contact with the flexible lumen.

12. The magnetic resonance imaging system of claim 8, wherein the magnetic resonance coil is at least partially formed from a conductive ink.

13. The magnetic resonance imaging system of claim 12, wherein the magnetic resonance coil is formed from a metal coupled to the conductive ink.

14. A method for magnetic resonance imaging of anatomical locations within a patient, the method comprising:
inserting a catheter into the patient, the catheter including
a flexible lumen supported on a distal end of the catheter that is configured to be expanded and contracted using a fluid introduced via a proximal end of the catheter,
a magnetic resonance coil formed on an exterior surface of the flexible lumen, the flexible lumen being contracted during insertion;
locating the catheter to a desired anatomical location within the patient;
expanding the flexible lumen; and
performing magnetic resonance imaging using a magnetic resonance imaging system.

15. The method of claim 14, further including matching and tuning the magnetic resonance coil based on the anatomical location of the catheter via a match and tune circuit that is disposed externally to the patient.

16. The method of claim 15, wherein the match and tune circuit is coupled to the magnetic resonance coil via a cable including at least one balun.

17. The method of claim 14, wherein expanding the flexible lumen includes expanding the magnetic resonance coil formed on the flexible lumen.

18. The method of claim 14, wherein inserting the catheter includes placing the catheter into a gastrointestinal tract of a patient.

19. The method of claim 14, wherein the magnetic resonance coil is at least partially formed from a conductive ink.

20. The method of claim 19, wherein the magnetic resonance coil is formed from a metal coupled to the conductive ink.
